(19) United States
(12) Patent Application Publication
(10) Pub. No.: US 2025/0380884 A1
(43) Pub. Date: Dec. 18, 2025

(54) AUDIOMETRY TEST METHOD AND ASSOCIATED ELECTRONIC DEVICE

(71) Applicant: MY MEDICAL ASSISTANT, Reims (FR)

(72) Inventor: Nicolas WALLAERT, Reims (FR)

(73) Assignee: MY MEDICAL ASSISTANT, Reims (FR)

(21) Appl. No.: 18/835,888

(22) PCT Filed: Feb. 6, 2023
(86) PCT No.: PCT/EP2023/052758

§ 371 Date: Feb. 10, 2025

(30) Foreign Application Priority Data
Feb. 8, 2022 (FR) FR2201113

(51) Int. Cl. A61B 5/12

(52) U.S. Cl. CPC A61B 5/123

(57) ABSTRACT

A method for audiometry testing of an ear comprising the repetition of the following steps:
Determination (S30) of a second sound with a second frequency and a second intensity from a Gaussian process,
Determination (S40), if present, of a third sound already applied to the ear having a third intensity, lower than the second intensity and higher than a second threshold and having a third frequency, the difference between the third frequency and the second frequency being lower than a frequency threshold, the third intensity being maximum among all the sounds whose frequency has a difference with the second frequency lower than the frequency threshold,
Application (S50) to the ear:
   A sound with the second frequency and the third intensity increased by a predefined increment, if the second intensity is greater than the second threshold, and if a third sound exists,
   Otherwise, second sound.

The invention concerns pure-tone audiometry.

Pure-tone audiometry provides a discrete measurement, via air- and bone-conduction, of a hearing threshold for a sound range extending, for example, from 125 to 8000 Hertz (as per standards, Hertz is referred to as Hz in the following) for conversational frequencies in air transmission mode, and from 250 to 6000 Hz in bone transmission mode. For high-frequency pure-tone audiometry, the sound range tested can be extended to 20000 Hz.

A series of sounds is applied to the ear under test, via air-conduction headphones or a bone-conduction vibrator, for example. The patient is asked to press a response button as soon as he or she hears a sound.

A Gaussian process has been proposed to determine the precise sounds to be presented to the patient, so as to quickly and accurately obtain the hearing threshold for the entire sound range.

For example, such an approach was published in Song X D, Wallace B M, Gardner J R, Ledbetter N M, Weinberger K Q, Barbour D L. “Fast, Continuous Audiogram Estimation using Machine Learning”. Ear Hear. 2015; 36 (6): e326-35.

There is a need to make such an approach applicable to a wide audience and for the entirety of the data collected during a pure-tone audiometry test (in air- and bone-conduction modes).

To this end, the invention concerns a method of audiometry testing of an ear (of a patient, in air- or bone-conduction) (for a sound range) (of course, the same audiometry test can also test the contralateral ear simultaneously. The hearing of the patient's two ears combined is then tested. In this way, the audiometry test according to the invention can be carried out in free-field mode), and comprises the following steps:
Determination of a sequence comprising:
   First sounds (within the sound range) applied to the ear,
   For each of the first sounds, initial information as to whether the first sounds are heard or not (by the patient),
Repeat the following steps a number of times until a condition is met:
   Determination of a second sound with a second frequency and intensity from a Gaussian process taking the sequence as observed data,
   Determination, if any, of a third sound of (i.e., in) the sequence having a third intensity below the second intensity and above a second threshold and (the third sound) having a third frequency, the difference between the third frequency and the second frequency being below a frequency threshold (the frequency threshold may be fixed or variable, for example, set at ½ octave), and the third intensity being maximum among all the sounds of the sequence and whose frequency has a difference from the second frequency that is below the frequency threshold,
   Application to the ear of a fourth sound with a fourth frequency and a fourth intensity, where:
      If the second intensity is greater than the second threshold, and a third sound is present, the fourth frequency is equal to the second frequency and the fourth intensity is equal to the third intensity increased by a predefined increment,
      If not (i.e., the second intensity is below the second threshold or there is no third sound), then the fourth sound is identical to the second sound,
   Acquisition of a fourth piece of information: whether or not the fourth sound is heard (by the patient),
   Updating the sequence with the fourth item of information and the fourth sound (in other words: the fourth item of information and the fourth sound are added to the sequence).

This avoids a too rapid increase in intensity beyond the second threshold around each frequency, and hence, prevents over-stimulation by taking into account the “loudness-recruitment” phenomena (abnormally rapid increase in loudness that can occur with sensorineural hearing loss). In this way, the audiometry test process according to the invention is suitable even in cases of severe hearing loss.

According to a known embodiment, the Gaussian process, defined from the observed data, probabilities of hearing each sound in the sound range and an uncertainty on these probabilities, and the second sound has a second frequency and a second intensity maximizing a reduction of this uncertainty.

According to one embodiment, the Gaussian process is implemented as described in Schlittenlacher J, Turner R E, Moore B C J. “Audiogram estimation using Bayesian active learning” (J Acoust Soc Am. 2018; 144 (1): 421). Notably the process has the following characteristics.

At the frequency level, for example, an exponential kernel is used to account for the fact that thresholds at adjacent frequencies are correlated, for example:

$${k}_{SE} ( x , {x}^{′} ) = {σ}^{2} exp ( - \frac{{( x - {x}^{′} )}^{2}}{2 {\text{?}}^{2}} )$$

$$\text{?} \text{indicates text missing or illegible when filed}$$

where the first term on the left (k_{SE}(x,x′)) corresponds to the square exponential kernel. The kernel is used to calculate a covariance matrix, which is then used to create functions. The term σ corresponds to the variance, i.e., the distance of the functions from the mean or the modulation depth, I corresponds to the length of the ripples, and x and x′ corresponds to all possible pairs of points.

In terms of intensity, for example, a linear kernel is used to account for the fact that the probability of a sound being heard increases with increasing intensity, for example:

$$k ( {x}_{i} , {x}_{j} ) = {σ}_{0}^{2} + {x}_{i} \cdot {x}_{j}$$

where the first term on the left (k(xᵢ,xⱼ)) corresponds to the linear kernel; σ₀ corresponds to the variance of the intercept of the line, i.e., a large value corresponds to a large variability on the intercepts; and xᵢ and xⱼ corresponds to all possible pairs of points.

The second sound, for example, is chosen to maximize the following function, which measures the mutual information between the expected response and the Gaussian process estimate:

$$I ( {y}^{\text{?}} ; \text{?} \vert {x}^{\text{?}} ) = H ( {y}^{\text{?}} \vert {x}^{\text{?}} , D ) - {E}_{θ ∼ p ( θ \vert D )} ( H [ {y}^{\text{?}} \vert {x}^{\text{?}} , θ ] ) ,$$

$$\text{?} \text{indicates text missing or illegible when filed}$$

where the first term on the right is the entropy of the expected response and the second term is the expected conditional entropy of the response given the estimated Gaussian process function; H is the Shannon entropy; D is the responses already obtained (i.e., the information in the sequence); x* is the frequency and signal intensity of the second sound; y* is the expected response; and θ is the Gaussian process function.

For example, the condition is met if:
The amplitude of the uncertainty interval estimated by the Gaussian process (the uncertainty interval is determined as the area covered by one standard deviation around the audiogram estimated by the Gaussian process, i.e., where the probability is 50%) is less than a threshold value (for example, 6 dB HL, or more generally a threshold value between 1 to 10 dB), or
The number of times exceeds a repetition threshold (for example, between 15 and 100 times).

According to one embodiment, the step of determining a sequence comprises repeating the following steps until a fifth frequency has taken on all the frequency values of a series of frequencies:
Determination of a fifth sound having the fifth frequency and a fifth intensity,
Application of the fifth sound to the ear,
Acquisition of a fifth piece of information, depending on whether or not the fifth sound is heard (by the patient),
The sequence is updated with the fifth sound and the fifth piece of information.

Alternatively, the sequence can be received or read from memory.

According to one embodiment, the step of determining a sequence comprises the first repetition of the following steps until a fifth frequency has taken on all frequency values of a frequency series:
Determination of a fifth sound having the fifth frequency and a fifth intensity,
A (second) repetition of the following (five) steps until the fifth sound has been heard once and not heard once, or until the fifth intensity reaches a maximum intensity, for example, 90 dB HL (i.e., decibels for hearing loss are noted as “dB HL”) or a minimum intensity, for example, −20 dB HL:
   Application of the fifth sound to the ear (210),
   Acquisition of a fifth piece of information, depending on whether or not the fifth sound is heard (by the patient),
   Updating the sequence with the fifth sound and the fifth piece of information
   If the fifth sound is heard, decrement the fifth intensity by one decrement value, for example, 20 dB HL.
   If the fifth sound is not heard, increment the fifth intensity by one incremental value, for example, by 10 dB HL.
Assign the fifth intensity by (i.e., the fifth intensity takes the next value):
   If a fifth sound was heard (by the patient) (during the second repetition of steps), a minimum intensity where the fifth sound was heard (at the fifth frequency) to which an intensity margin is added, for example, 10 dB HL.
   If the fifth sound has not been heard (by the patient) (during the second repetition of steps), the maximum intensity, for example, 90 dB HL.
Increase or decrease the fifth frequency by one frequency increment, (so that the fifth frequency takes on all frequency values in the frequency series), for example, an increase or decrease of 500 Hz.

Alternatively, the audiometry test method is performed in bone-conduction (i.e., the fourth sound is applied to the ear in bone-conduction) and comprises, prior to the step of determining a sequence:
A determination of an air-conduction audiogram comprising an air intensity for each frequency in a frequency series (the air intensity for each frequency is the minimum intensity at which sound is heard at that frequency),
Initialization of a state to a first value (the state is stored in memory, for example),
   and wherein the step of determining a sequence comprises the first repetition of the following steps until a fifth frequency has taken all frequency values of the frequency series, the fifth frequency being initialized to the first frequency of the frequency series:
If the state has the first value:
   Determination of a fifth sound with the fifth frequency, and a fifth intensity equal to the air intensity for the fifth frequency in the air-conduction audiogram, to which an additional intensity is added, for example, 5 dB,
   Application of the fifth sound by bone-conduction to the ear (210) (masking of the fifth sound can be applied to the contralateral ear),
   Acquisition of a fifth piece of information as to whether the fifth sound is heard or not,
   The sequence is updated with the fifth sound and the fifth piece of information,
   If the fifth sound is heard, the state is assigned a second value.
If the state has the second value:
   A repetition of the following (five) steps until the fifth sound has been heard once and not heard once, or until the fifth intensity reaches maximum intensity, or minimum intensity:
      Application of the fifth sound to the ear (210),
      Acquisition of a fifth piece of information, depending on whether the fifth sound is heard or not,
      The sequence is updated with the fifth sound and the fifth piece of information,
      If the fifth sound is heard, decrement the fifth intensity by one decrement value.
      If the fifth sound is not heard, increment the fifth intensity by one incremental value.
   Allocation of the fifth intensity by:
      If the fifth sound has been heard, a minimum intensity where the fifth sound has been heard to which an intensity margin is added,
      If the fifth sound has not been heard, maximum intensity,
Increase the fifth frequency by one frequency increment (so that the fifth frequency takes on all the frequency values in the frequency series).

When the fifth frequency is an end frequency (for example, 8000 Hz and 125 Hz are end frequencies when the frequency series consists of the following frequencies: 1000, 1500, 2000, 3000, 4000, 6000, 8000, 750, 500, 250 and 125 Hz) of the frequency series, the increment value (and/or the decrement value, respectively) can take on a lower value, for example 10 dB HL, than the decrement value (and/or the decrement value, respectively) when the frequency is another frequency (than an end frequency). When the fifth intensity exceeds a fifth threshold, for example, 80 dB, the increment value takes on a lower value, for example, 5 dB HL, than the increment value (used) when the fifth intensity is below the fifth threshold.

This avoids over-stimulation through loudness recruitment for patients that may occur during the sequence determination stage.

The frequency series can be made up of the following frequencies: 1000, 2000, 4000, 8000, 500, and 250 Hz. However, a series with a larger number of frequencies has the advantage of facilitating the correction of subject response errors (e.g., when a patient does not indicate that he has heard a sound even though he has). Preferably, the frequency series consists of the following frequencies: 1000, 1500, 2000, 3000, 4000, 6000, 8000, 750, 500, 250 and 125 Hz for air-conduction and 1000, 1500, 2000, 3000, 4000, 6000, 750, 500 and 250 Hz for bone-conduction.

According to one embodiment, during the step of determining the second sound, the second sound is determined, from the Gaussian process, in a range of sound frequencies (in other words, the Gaussian process defines probabilities of hearing each sound in the range of sound frequencies and an uncertainty is assigned to these probabilities, and the second sound maximizes a reduction of this uncertainty), and the range of sound frequencies excludes a range of exclusion frequencies where no sound (i.e., no emitted sound, having a frequency in the range of exclusion frequencies) has been heard (by the patient) during the first repetition (i.e., no emitted sound with a frequency in the exclusion frequency range was heard (by the patient) during the first repetition). For example, within the range of sound frequencies, during the first repetition, one sound is heard for each frequency of the series of sound frequencies within the range of sound frequencies. In one embodiment, the sound frequency range retains the boundaries of this exclusion range.

This reduction in bandwidth is achieved in such a way as to enable the process according to the invention to converge more rapidly, and to avoid an unsuccessful search for thresholds on parts of the spectrum for which no additional information could be obtained due to a cochlear dead zone or the limitation imposed by the maximum power deliverable by the audiometry equipment used, which will not allow the threshold to be estimated.

The process according to the invention can be carried out (in other words, implemented) by an electronic audiometry testing device. The electronic device may comprise a central electronic unit (for example, included in a cell phone or touch-screen tablet) and headphones or inserts, or loudspeakers for applying sounds to the ear and masking the contralateral ear in air-conduction mode. For bone-conduction mode, one or more vibrators (not shown) are used. Information as to whether a sound is heard or not can be acquired by a button that the patient presses when a sound is heard, or by voice command or image detection.

The invention also relates to an electronic audiometry testing device configured to implement the steps of the process according to the invention.

The invention also relates to a computer program comprising instructions, executable by a microprocessor or microcontroller, for implementing the method according to the invention.

The features and benefits of the electronic device and the computer program are identical to those of the process, so they are not repeated here.

An element such as an electronic audiometry test device, central processing unit or other element is “configured to” perform a step or operation, by virtue of the fact that the element comprises means for (in other words, “is configured to” or “is adapted to”) performing the step or operation. These are preferably electronic means, such as a computer program, stored data and/or specialized electronic circuits.

When a step or operation is carried out by such an element, this generally implies that the element has means for (in other words, “is shaped to” or “is adapted to”) carrying out the step or operation. These may include electronic means, such as a computer program, stored data and/or specialized electronic circuits.

Other features and advantages of the present invention will become clearer on reading the following detailed description including modes of realization of the invention given by way of non-exhaustive examples and illustrated by the appended drawings, in which:

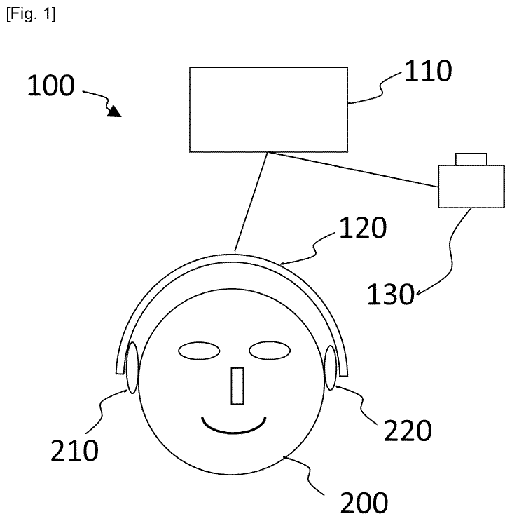

FIG. 1 shows an electronic device according to one embodiment of the invention.

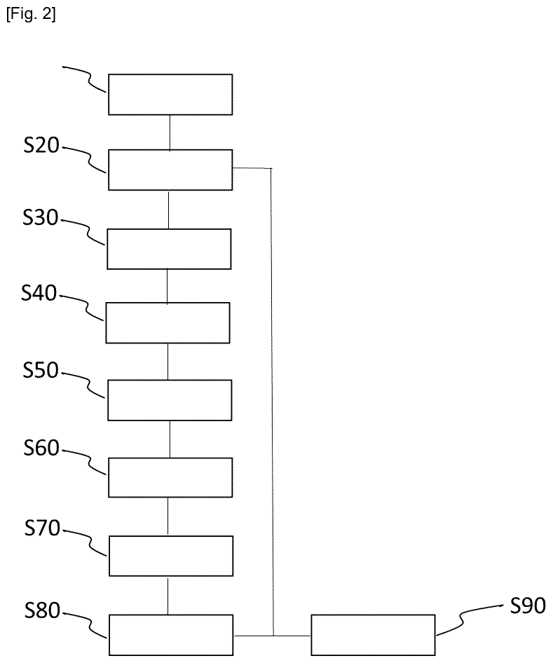

FIG. 2 shows the process according to the invention, in one example embodiment, implemented by the electronic device shown in FIG. 1.

### DETAILED DESCRIPTION OF AN EMBODIMENT OF THE INVENTION

Referring to FIGS. 1 and 2, In step S10 the process begins with the determination of a sequence, the determination of the sequence comprising the first repetition of the following steps until a fifth frequency has assumed all values (in other words, every value) of frequency of a frequency series:
Determination of a fifth sound with a fifth frequency, for example, 1000 Hz, and a fifth intensity, for example, 60 dB HL,
Repeating the following steps until the fifth sound has been heard once and not heard once, or until the fifth intensity reaches a maximum intensity, for example, 90 dB HL or a minimum intensity, for example, −20 dB HL:
   Application of the fifth sound to ear 210,
   Acquisition of a fifth the information as to whether the fifth sound is heard or not (by the patient),
   Updating the sequence with the fifth sound and the fifth piece of information
   If the fifth sound is heard, decrement the fifth intensity by one decrement value, for example, 20 dB HL.
   If the fifth sound is not heard, increment the fifth intensity by one incremental value, for example, by 10 dB HL.
Allocation of the fifth intensity by:
   If a fifth sound has been heard (by the patient) (during the second repetition of steps), a minimum intensity where the fifth sound has been heard (at the fifth frequency) to which an intensity margin is added, for example, 10 dB HL. If, in the above example, the fifth sound is heard at 1000 Hz and 60 dB HL and not heard at 50 dB HL, then the fifth intensity at this stage takes on the value of 70 dB HL.
   If the fifth sound has not been heard (by the patient) (during the second repetition of the steps), a maximum intensity, for example, 90 dB HL.
Increase or decrease the fifth frequency by one frequency increment (preferably so that the fifth frequency has taken on all the frequency values of a frequency series), for example, 500 Hz. In the above example, the fifth frequency is thus increased to 1500 Hz. The next sound applied to the ear is therefore at 1500 Hz and 70 dB HL.

Alternatively, the audiometry test process is carried out in bone-conduction and comprises, prior to the step of determining a sequence, a determination of an air-conduction audiogram comprising a threshold of 40 dB at 1000 Hz and 45 dB at 1500 Hz. The patient is first made to hear a sound at 1000 Hz and 45 dB. If the sound is not heard, bone-conduction is assumed to be identical to air-conduction at 1000 Hz. The patient is then played a sound at 1500 Hz and 50 dB. If the sound is heard, the test continues as described above in step S10, taking the 50 dB intensity and 1500 Hz frequency sound as the fifth sound.

For example, when the fifth frequency is an end frequency (for example, the frequencies 8000 Hz and 125 Hz are end frequencies in air-conduction mode when the frequency series consists of the following frequencies: 1000, 1500, 2000, 3000, 4000, 6000, 8000, 750, 500, 250 and 125 Hz) of the frequency series, the increment value (and/or the decrement value, respectively) takes on a lower value, for example 10 dB HL, than the increment value (and/or the decrement value, respectively) when the frequency is another frequency (than an end frequency).

When the fifth intensity exceeds a fifth threshold, for example, 80 dB HL, the increment value takes on a lower value, for example, 5 dB HL, than the increment value when the fifth intensity is below the fifth threshold.

The frequency series comprises, for example, the following frequencies: 1000, 1500, 2000, 3000, 4000, 6000, 8000, 750, 500, 250 and 125 Hz.

For example, in step S10, during the first repetition, no sound is heard by patient 200 at 6000 Hz and 8000 Hz. Instead, a sound is heard at frequencies 1000, 1500, 2000, 3000, 4000, 750, 500, 250 and 125 Hz.

Step S20 checks whether a condition has been met. The condition is met if:
The amplitude of an interval estimated by the Gaussian process is below a threshold value (for example, 6 dB HL),
The number of times exceeds a repetition threshold (for example, between 15 and 100 times).

If the condition is met, the process ends at step S90.

If the condition is not met, then the following steps are performed:
In step S30, determination of a second sound having a second frequency, for example, 4500 Hz, and a second intensity, for example, 105 dB HL, from a Gaussian process taking the sequence as observed data in a sound frequency range between 125 Hz and 6000 Hz (since in step S10, during the first repetition, no sound for example is heard by patient 200 at 6000 Hz and 8000 Hz).
At step S40, determination of whether there is a third sound in the sequence having a third intensity, for example, 90 dB HL, lower than the second intensity and higher than a second threshold, for example, equal to 80 dB HL, and a third frequency, for example, 4000 Hz, for which the difference between the third frequency and the second frequency is lower than a frequency threshold, for example, equal to 500 Hz, and for which the third intensity is maximum among all the sounds in the sequence whose frequency has a difference with the second frequency lower than the frequency threshold (for example, the sequence comprises, between 4000 Hz and 6000 Hz, a sound at 4000 Hz and 90 dB HL an unheard sound at 6000 Hz. The third sound is the sound at 4000 Hz and 90 dB HL),
In step S50, a fourth sound having a fourth frequency and a fourth intensity is applied to ear 210, where:
   If the second intensity is greater than the second threshold, for example, equal to 80 dB HL, and if there is a third sound, the fourth frequency is equal to the second frequency, (in our example, 4500 Hz), and the fourth intensity is equal to the third intensity (in our example, 90 dB HL) increased by a predefined increment, for example, 5 dB HL. Therefore, the fourth intensity is equal to 95 dB HL.
   If not (i.e., the second intensity is below the second threshold or there is no third sound), then the fourth sound is identical to the second sound.
At step S60, acquisition of a fourth item of information, depending on whether or not the fourth sound is heard (by the patient).
At step S70, the sequence is updated with the fourth item of information and the fourth sound.
At step S80, the process continues with step S20.

For example, the Gaussian process is implemented as described in Schlittenlacher J, Turner R E, Moore B C J. “Audiogram estimation using Bayesian active learning” (J Acoust Soc Am. 2018; 144 (1): 421). Notably the process has the following characteristics.

The process according to the invention can be carried out (in other words, implemented) by an electronic device 100 of audiometry testing. The electronic device 100 may comprise a central unit 110 and headphones 120 for applying sounds to the ear 210 and masking the contralateral ear 220 in air transmission of a patient 200. In bone-conduction mode, one or more vibrators (not shown) are used in combination with air-conduction headphones for masking the contralateral ear. Information as to whether a sound is heard or not can be acquired by means of a button 130 which the patient presses when a sound is heard.

1. Method for audiometry testing of an ear (210), characterized in that it comprises the following steps:
Determination (S10) of a sequence comprising:
From the first sounds applied to the ear (210),
For each of the first sounds, initial information as to whether the first sounds are heard or not,
Repeat the following steps a number of times until a condition is met (S20):
Determination (S30) of a second sound having a second frequency, and a second intensity, from a Gaussian process taking the sequence as observed data,
Determination (S40), if any, of a third sound in the sequence having a third intensity, lower than the second intensity and higher than a second threshold, and having a third frequency, the difference between the third frequency and the second frequency being lower than a frequency threshold, and the third intensity being maximum among all the sounds in the sequence whose frequency has a difference with the second frequency lower than the frequency threshold,
Application (S50) to the ear (210) of a fourth sound having a fourth frequency and a fourth intensity, whereby:
If the second intensity is greater than the second threshold, and if a third sound exists, the fourth frequency is equal to the second frequency, and the fourth intensity is equal to the third intensity increased by a predefined increment,
If not, then the fourth sound is identical to the second sound.
Acquisition (S60) of fourth information on whether or not the fourth sound is heard,
Update (S70) of the sequence based on the fourth item of information and the fourth sound.

2. Audiometry test method according to the preceding claim in which the condition is fulfilled if:
An interval amplitude estimated by the Gaussian process is below a threshold value, or
The number of times exceeds a repetition threshold.

3. An audiometry test method according to any one of claim 1 or 2 in which the step of determining a sequence comprises repeating the following steps until a fifth frequency has taken on all the frequency values of a series of frequencies:
Determination of a fifth sound with a fifth frequency and a fifth intensity,
Application of the fifth sound to the ear (210),
Acquisition of a fifth piece of information, depending on whether or not the fifth sound is heard (by the patient),
The sequence is updated with the fifth sound and the fifth piece of information.

4. Audiometry test method according to any one of claim 1 or 2 in which the step of determining a sequence comprises the first repetition of the following steps until a fifth frequency has taken on all the frequency values of a series of frequencies:
Determination of a fifth sound with a fifth frequency and a fifth intensity,
Repeat the following steps until the fifth sound has been heard once and not heard once, or until the fifth intensity reaches maximum intensity, or minimum intensity:
Application of the fifth sound to the ear (210),
Acquisition of a fifth piece of information as to whether the fifth sound is heard or not,
The sequence is updated with the fifth sound and the fifth piece of information,
If the fifth sound is heard, decrement the fifth intensity by one decrement value.
If the fifth sound is not heard, increment the fifth intensity by one incremental value.
Allocation of the fifth intensity by:
If the fifth sound has been heard, a minimum intensity where the fifth sound has been heard to which an intensity margin is added,
If the fifth sound has not been heard, set the intensity to maximum,
Increase or decrease the fifth frequency by one frequency increment.

5. Audiometry test method according to the preceding claim, in which, when the fifth frequency is an end frequency of the frequency series, the increment value takes on a lower value than the increment value when the frequency is another frequency.

6. Audiometry test method according to any one of claim 1 or 2, in which the audiometry test is performed in bone-conduction and comprising, prior to the step of determining a sequence:
A determination of an air-conduction audiogram comprising an air intensity for each frequency in a frequency series (the air intensity for each frequency is the minimum intensity at which sound is heard at that frequency),
Initialization of a state to a first value (for example, the state is stored in memory)
and wherein the step of determining a sequence comprises the first repetition of the following steps until a fifth frequency has taken all frequency values of the frequency series, the fifth frequency being initialized to the first frequency of the frequency series:
If the state has the first value:
Determination of a fifth sound with the fifth frequency, and a fifth intensity equal to the air intensity for the fifth frequency in the air-conduction audiogram, to which an additional intensity is added,
Application of the fifth bone-conduction sound to the ear (210),
Acquisition of a fifth piece of information as to whether the fifth sound is heard or not,
If the fifth sound is heard, the state is assigned a second value.
If the state has the second value:
Repeat the following steps until the fifth sound has been heard once and not heard once, or until the fifth intensity reaches maximum intensity, or minimum intensity:
Application of the fifth sound to the ear (210),
Acquisition of a fifth piece of information as to whether the fifth sound is heard or not,
The sequence is updated with the fifth sound and the fifth piece of information,
If the fifth sound is heard, decrement the fifth intensity by one decrement value.
If the fifth sound is not heard, increment the fifth intensity by one incremental value.
Allocation of the fifth intensity as follows:
If the fifth sound has been heard, a minimum intensity where the fifth sound has been heard to which an intensity margin is added,
If the fifth sound has not been heard, set the intensity to maximum,
Increase the fifth frequency by one frequency increment.

7. Audiometry test method according to any one of claims 4 to 6, in which, when the fifth intensity exceeds a fifth threshold, the increment value takes on a lower value than the increment value when the fifth intensity is below the fifth threshold.

8. Audiometry test method according to any one of claims 4 to 7 in which, during the step of determining the second sound, the second sound is determined, from the Gaussian process, in a sound frequency range, and the sound frequency range excludes an exclusion frequency range where no sound was heard during the first repetition.

9. Audiometry test method according to any one of claims 3 to 8 in which the frequency series consists of the following frequencies: 1000, 1500, 2000, 3000, 4000, 6000, 8000, 750, 500, 250 and 125 Hz in air-conduction and 1000, 1500, 2000, 3000, 4000, 6000, 750, 500 and 250 Hz in bone-conduction.

10. Electronic audiometric testing device (100) configured to implement the steps of the process according to any one of claims 1 to 9.

11. Computer program comprising instructions, executable by a microprocessor or microcontroller, for implementing the process according to any one of claims 1 to 9.
